(19) United States
(12) Patent Application Publication
(10) Pub. No.: US 2008/0097179 A1
(43) Pub. Date: Apr. 24, 2008

(54) FEEDING TUBE SYSTEM WITH REFLUX FLUID SENSING

(76) Inventor: Susan H. Russo, Lincoln City, OR (US)

Correspondence Address: PAUL J. FORDENBACHER;SILICON FOREST PATENT GROUP, 11876 NW TYLER CT., PORTLAND, OR, 97229, US

(21) Appl. No.: 11/428,545

(22) Filed: Jul. 3, 2006

(51) Int. Cl. A61B 5/00; A61M 25/00

(52) U.S. Cl. 600/343; 604/264

(57) ABSTRACT

Embodiments of a feeding tube system of the present invention provide a feeding tube that provides for monitoring whether it has detected reflux fluid which could potentially lead to serious medical conditions, such as, in the case of a nasogastric tube used for enteral feeding, aspiration of tube feeding into the patient's lungs. The reflux event is made apparent to medical staff and automated systems provide to take action, such as, in the case of enteral feeding, to shut off the delivery of enteral feeding to the patient. This allows remedial measures to be taken so that the associated morbidity and mortality can be prevented. The methods and apparatus are readily acceptable and easy to use by the medical staff, safe for the patient, and inexpensive to manufacture.

### FIELD OF THE INVENTION

The present invention is related to medical equipment, and more particularly, to methods and apparatus for feeding tubes.

### BACKGROUND

A nasogastric tube is one type of enteral feeding tube used to provide enteral nutritional support to a patient with a functional gastrointestinal tract who cannot meet their caloric needs by taking in foods orally. The nasogastric tube is commonly constructed of a flexible material, such as, but not limited to polyvinyl chloride. The nasogastric tube has a tube proximal end, a tube distal end, and at least one lumen there between. There are several nasogastric tubes available on the market for administering enteral nutrition to patients which vary in length, composition, diameter and number of lumens.

The nasogastric tube is inserted through the nose or mouth of the patient and advanced into the stomach or duodenum. The distal end of the nasogastric tube that resides in the stomach or duodenum has apertures through the walls of the tube that allow the enteral nutrition to exit the lumen and enter the patient. The proximal end of the nasogastric tube that resides outside of the body is connected to one of several devices that allow for the administration of enteral nutrition into the lumen and thus into the patient.

A syringe can be connected to the proximal end of the nasogastric tube to give a bolus of enteral nutrition. The enteral nutrition can also be placed into a reservoir bag that is connected to a tube that couples to the proximal end of the nasogastric tube. This allows the enteral nutrition to travel from the reservoir bag to the patient via gravity. The tube of the reservoir bag can also pass through a feeding pump that allows for the delivery of the enteral nutrition to the patient at a specific volume per hour.

Enteral tube feeding has been proven to promote nitrogen retention, accelerate wound healing, and improve overall nutritional status. Enteral tube feeding is favored over intravenous feeding because it helps to maintain intestinal integrity and has a lower infection risk. One of the major drawbacks of enteral tube feeding, however, is the possibility of aspiration of gastric contents into the lungs.

Aspiration is one of the most serious and potentially life-threatening complications of enteral tube feeding. This complication is documented to occur in nearly one percent (0.8%) of the patients receiving a course of enteral nutrition. Aspiration is the condition wherein the enteral nutrition inadvertently enters the esophagus and then subsequently into the lungs. One cause of aspiration is the reflux fluid of gastric contents into the esophagus and subsequently into the lungs despite the distal end of the enteral feeding tube remaining in its correct position in the stomach or duodenum. There are many causes for the reflux fluid of the enteral nutrition into the esophagus. They may include the presence of the feeding tube itself, medications, surgical procedures, neuromuscular problems, delayed gastric emptying, poor esophageal sphincter tone, rapid rate of enteral infusion, and intestinal obstruction.

The consequences of enteral nutrition or gastrointestinal secretions entering the lungs can range from coughing and wheezing to infection and respiratory failure. The effect of aspiration on the patients depends on the volume, pH, particle size, composition and microbial content, among others, of the aspirated material and the health of the patient. In addition to the possible human suffering incurred with such a complication, expenses on the order of thousands of dollars per event per day can be generated by antibiotic costs, intensive care and respiratory support.

There are many protocols used in the clinical environment aimed at preventing aspiration. These include surveillance of nasogastric tube placement, monitoring gastrointestinal residual volume, elevating the head of the bed, using medications to enhance gastric emptying, and using smaller diameter nasogastric tubes. Nevertheless, aspiration still occurs in patients receiving enteral nutrition with currently-available enteral feeding tubes, including nasogastric tubes.

In patients receiving enteral nutrition through nasogastric tubes, the solutions to-date for preventing reflux have been ineffective for the most part or potentially injurious. Moreover, complicated and impractical “solutions” annoy the medical staff, generate extra costs, and place patients at risk. The current trend in medicine towards managed care will put pressure on hospitals to reduce complication rates while keeping costs down. Cost-cutting measures lead to leaner staffing and therefore, less supervision of patients with nasogastric tubes. Not only will reflux of enteral nutrition from nasogastric tubes increase in this setting but the discovery of reflux fluid will be protracted making aspiration more likely and patient morbidity more severe.

Methods and apparatus for the detection of gastric reflux are needed in the art to provide opportunity for early intervention in order to thwart potential medical complications. The detection should be easily sensed by the medical staff or by automated systems to allow for remedial measures to be taken so that the associated morbidity and mortality can be prevented. The methods and apparatus should be readily acceptable and easy to use by the medical staff, safe for the patient, and inexpensive to manufacture.

### SUMMARY

The present invention is related to detecting fluid in a cavity within the body. It is understood that the term “medical tubes” is used in a general sense and includes those tubes having one end internal and one end external to the body. Examples of medical tubes include, but are not limited to, orogastric tubes and nasogastric tubes. The description provided below includes reference to enteral feeding tubes and detection of gastric reflux fluid in the esophagus, but the present invention is not limited thereto.

A system including a reflux-sensing feeding tube and sensor electronics is provided in accordance with an embodiment of the present invention. The reflux-sensing feeding tube comprises a feeding tube and reflux sensor apparatus coupled to the feeding tube in a predetermined location. The reflux sensor apparatus is adapted to communicate with the sensor electronics so as to provide information dependent on the detected reflux fluid. Reflux fluid, that is, the fluid that is refluxed into the esophagus and potentially into the lungs, is sensed by the sensor electronics. The sensor electronics interprets state-data as that reflux fluid has been detected and responds in a predetermined way, such as, but not limited to, triggering an alarm and turning off a process.

In accordance with an embodiment of the present invention, a feeding tube system comprises sensor electronics and an electrode-tagged feeding tube comprising a feeding tube, and wherein the reflux sensor apparatus is an electrode apparatus coupled thereto. The electrode apparatus includes spaced-apart electrodes adapted to close an electrical circuit upon the presence of reflux fluid therebetween, wherein the closed circuit is detected by the sensor electronics. The system operates on the premise that reflux fluid is generally electrically conductive and when it comes between and bridges two electrodes, the reflux fluid closes the electrical circuit between the electrodes.

When the electrode-tagged feeding tube is properly placed within the patient's body and there is no reflux fluid in the patient's esophagus, the electrical circuit between two electrodes is open. When reflux fluid is present in the patient's esophagus, the reflux fluid will also be in the sensor lumen containing the electrodes, thus completing and closing the electrical circuit between the two electrodes. The sensor electronics is adapted to detect the condition of the electrical circuit, either open or closed, and provides a response suitable for a particular purpose.

In accordance with an embodiment of the present invention, a feeding tube system comprises sensor electronics and an optical-tagged feeding tube comprising a feeding tube, and wherein the reflux sensor apparatus is a light sensor apparatus coupled thereto. The light sensor apparatus includes spaced-apart light sources and light sensors, the light sensors adapted to detect light from the light sources and transmit the light to the sensor electronics. The system operates on the premise that reflux fluid is generally opaque and when it comes between a light source and a light sensor, the reflux fluid will block the reception of light by the light sensor.

When the optical-tagged feeding tube is properly placed within the patient's body and there is no reflux fluid in the patient's esophagus, the light sensor detects a first intensity of light from the light source. When reflux fluid is present in the patient's esophagus, the reflux fluid blocks light from reaching the light sensor, the light sensor detecting a reduced intensity of light from the light source. The sensor electronics is adapted to detect the intensity of the light received by the light sensor apparatus and provide a response suitable for a particular purpose.

Embodiments of a feeding tube system of the present invention provide a feeding tube system that continuously monitors whether a reflux event has taken place which could potentially lead to clinical problems, such as aspiration into the patient's lungs associated with using a nasogastric feeding tube, by way of example. The reflux event is made apparent to medical staff and automated systems are provided to shut off the delivery of enteral nutrition to the patient. This allows remedial measures to be taken so that the associated morbidity and mortality can be prevented. The methods and apparatus are readily acceptable and easy to use by the medical staff, safe for the patient, and inexpensive to manufacture. Other embodiments are as presented herein.

### BRIEF DESCRIPTION OF THE DRAWINGS

Like reference numbers generally indicate corresponding elements in the figures.

FIG. 1 is a side perspective view of a feeding tube system including a reflux-sensing feeding tube and sensor electronics, in accordance with an embodiment of the present invention;

FIG. 2 is a front partial cut-away view showing the feeding tube system relative to a patient, in accordance with an embodiment of the present invention;

FIG. 3 is a front partial cut-away view showing the feeding tube system relative to a patient, in accordance with an embodiment of the present invention;

FIG. 4 is a side partial cut-away view showing a feeding tube system comprising an electrode-tagged feeding tube, in accordance with an embodiment of the present invention;

FIG. 5 is a side perspective view of an electrode-tagged feeding tube, in accordance with and embodiment of the present invention;

FIG. 6 is a side perspective view of an electrode-tagged feeding tube comprising a feeding lumen and sensing lumen in parallel arrangement, in accordance with an embodiment of the present invention;

FIG. 7 is a side perspective view of an electrode-tagged feeding tube comprising a feeding lumen and sensing lumen in parallel arrangement, in accordance with another embodiment of the present invention;

FIG. 8 is a side perspective view of an optical-tagged feeding tube in accordance with an embodiment of the present invention;

FIG. 9A is a cross-sectional view of a feeding tube further comprising a sensor lumen that extends from the tube proximal end to the tube distal end, in accordance with an embodiment of the present invention;

FIG. 9B is a perspective view of a light guide sheet, in accordance with an embodiment of the present invention;

FIG. 9C is a perspective view of a gap showing an aperture in the light guide sheet so as to allow fluid to enter and come between the light emitting LG distal end and the light receiving LG distal end; and

FIGS. 10A and 10B are side perspective and end views, respectively, of an optical-tagged feeding tube wherein the reflux sensor apparatus comprises one or more pairs of light guides, in accordance with an embodiment of the present invention.

### DETAILED DESCRIPTION

References will now be made to embodiments illustrated in the drawings and specific language which will be used to describe the same. It will nevertheless be understood that no limitation of the scope of the invention is thereby intended, such alterations and further modifications in the illustrated devices, as such further applications of the principles of the invention as illustrated therein as being contemplated as would normally occur to one skilled in the art to which the invention relates.

Embodiments in accordance with the present invention relate to detecting reflux fluid of enteral nutrition during the use of a feeding tube within a patient's body. It is understood that the term “feeding tubes” is used in a general sense and include those tubes adapted to have one end internal and one end external to the body. Examples of feeding tubes include, but are not limited to, orogastric tubes and nasogastric tubes.

It is appreciated that the detection of reflux fluid is beneficial for other medical procedures and purposes which are within the scope of the present invention. Other medical procedures and purposes include, but are not limited to, the detection of reflux fluid during a procedure wherein a medical tube is used to evacuate the stomach of its contents, such as when a patient has an intestinal blockage or a gastrointestinal bleed. The sensing of reflux fluid in the esophagus would let the medical staff know that the suction used to evacuate the stomach is not operating correctly and reflux of stomach contents is occurring into the esophagus.

The description provided below includes reference to nasogastric tubes and detection of reflux fluid while using a nasogastric tube. It is understood that the description below is provided by way of example and is not limited to the described applications.

Reflux fluid is defined herein to include any fluid that enters the esophagus and has the potential to cause aspiration. Reflux fluid includes, but is not limited to, enteral nutrition that has been delivered to the stomach but is subsequently refluxed into the esophagus. Reflux fluid also includes enteral nutrition that is delivered to the esophagus and fails to enter the stomach, but backs up to pose an aspiration hazard. Reflux fluid also includes any stomach fluid that backs up into the esophagus.

Feeding tubes come in a variety of configurations, including single lumen and multi-lumen configurations. The one or more lumens can extend the entire length of the feeding tube, or one or more of the lumens can terminate at a predetermined distance from one of the feeding tube ends. For example, there are known nasogastric feeding tubes that have side apertures provided at predetermined distances from the tube distal end that allow fluid to pass between the lumen and the body cavity. For example, there are known feeding tubes that have a vent lumen and a feeding lumen that extend from a proximal end of the feeding tube but not through to the distal end of the feeding tube; i.e., a predetermined distance from the distal end. Side apertures are provided a predetermined distance from the tube distal end that allows air to pass from the body cavity to the vent lumen.

In the description, reference is made to a tube distal portion which includes a tube distal end, and a tube proximal portion which includes a tube proximal end. The tube distal portion is that portion of the tube that is advanced into the patient's body and the tube proximal portion is that portion of the tube that remains external to the patient's body. The tube proximal end is adapted to couple with apparatus suitable for which the feeding tube is used.

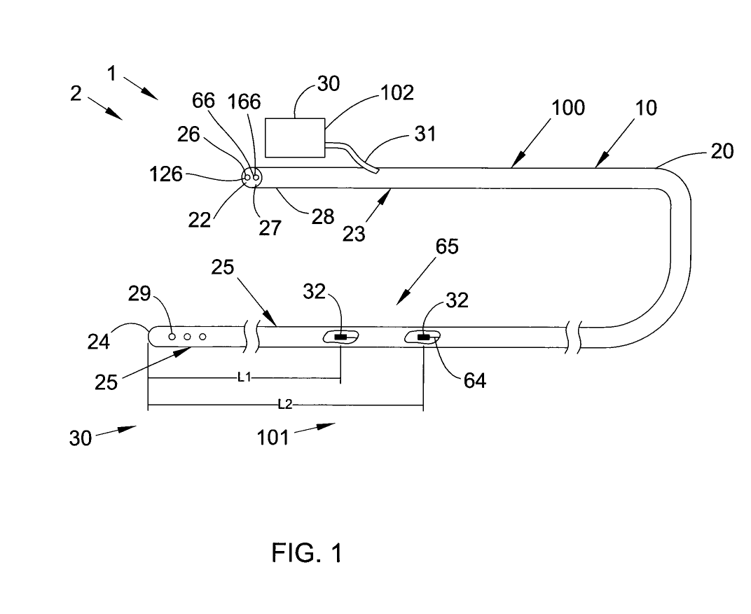

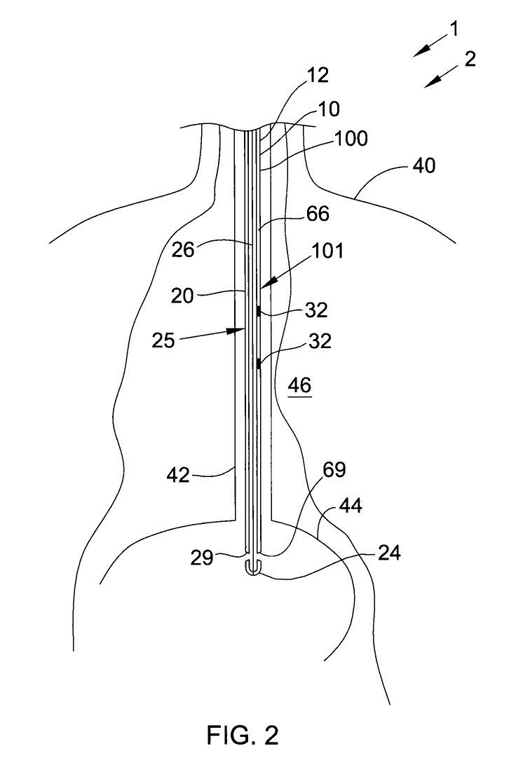

FIG. 1 is a side view of a feeding tube system 1 including a reflux sensing feeding tube 100 and sensor electronics 102, in accordance with an embodiment of the present invention. FIG. 2 is a front partial cut-away view showing the feeding tube system 1 relative to a patient 40, in accordance with an embodiment of the present invention. The reflux sensing feeding tube 100 comprises a feeding tube 20 and reflux sensor apparatus 101 coupled to the feeding tube 20 in a predetermined location. The reflux sensor apparatus 101 is adapted to communicate with the sensor electronics 102 so as to provide information dependent on the detection of reflux fluid into the feeding tube 20. Reflux fluid into the feeding tube 20 is detected by the reflux sensor apparatus 101 which communicates a signal to the sensor electronics 102. The sensor electronics 102 interprets the signal as an event that reflux fluid has entered the feeding tube 20 and responds in a predetermined way, such as, but not limited to, triggering an alarm and turning off a process.

Referring again to FIGS. 1 and 2, a feeding tube system 2 is provided wherein the reflux-sensing feeding tube 100 is an electrode-tagged feeding tube 10 comprising a feeding tube 20 and wherein the reflux sensor apparatus 101 is one or more electrodes 32, and the sensor electronics 102 is electrode sensor electronics 30, in accordance with an embodiment of the present invention. The feeding tube 20 comprises an elongated tubular member comprising a tube proximal end 22, a tube distal end 24, and a feeding lumen 26 and a sensor lumen 66 therebetween. The feeding tube 20 further comprises a tube wall 27 defined by the feeding lumen 26 and sensor lumen 66 and a tube outer surface 28. The feeding tube 20 further comprises a tube distal portion 25, including the tube distal end 24. The tube distal portion 25 is that portion of the feeding tube 20 that typically resides within the patient 40 when in use. The feeding tube 20 further comprises a tube proximal portion 23, including the tube proximal end 22. The tube proximal portion 23 is that portion of the feeding tube 20 that typically resides outside of the patient 40 when in use.

In an embodiment in accordance with a method of the present invention, an electrode-tagged nasogastric tube 12 is positioned within the patient 40 by passing the tube distal end 24 through the nose or oral pathway, through the esophagus 42 and disposed in either the stomach 44 or duodenum.

The feeding tube 20 further comprises at least one feeding lumen aperture 29 adjacent the tube distal end 24 in fluid communication with the feeding lumen 26. The feeding lumen 26 includes a feeding lumen inlet 126 adjacent the feeding tube proximal end 22. The feeding lumen 26 is adapted to accept a fluid, such as, but not limited to, enteral nutrition, at the feeding lumen inlet 126 and carry the fluid to the feeding lumen aperture 29 where the fluid exits the feeding lumen 26 and is delivered to the patient's stomach 44 or duodenum.

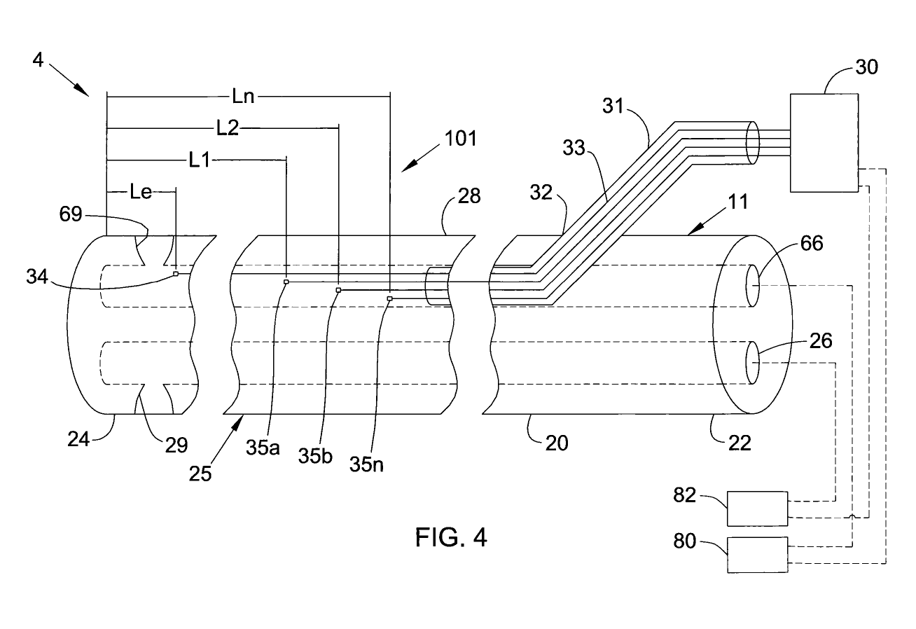

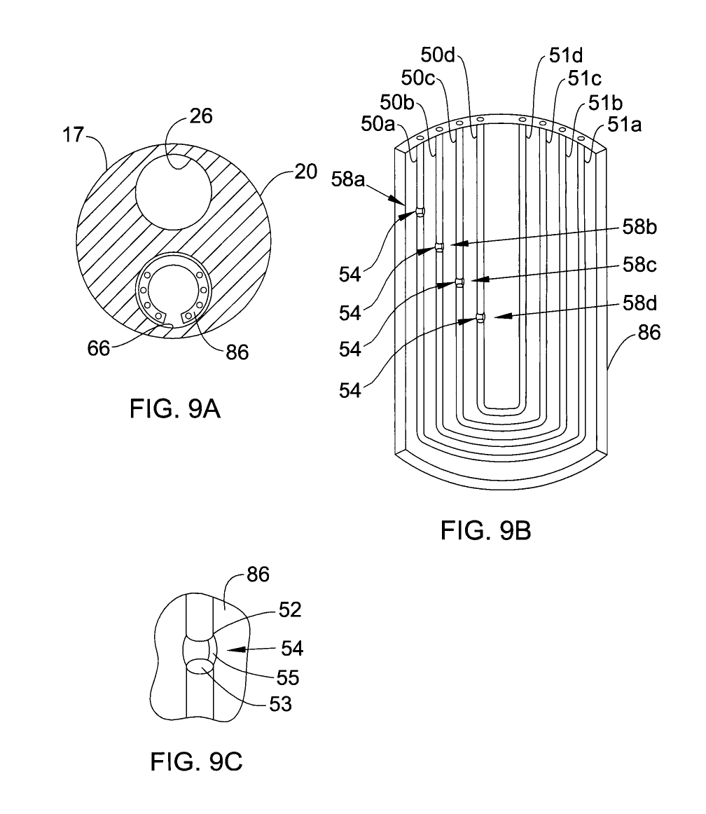

It is appreciated that a number of feeding lumen aperture configurations can be incorporated into the feeding tube 20 suitable for a particular purpose to deliver the fluid from the feeding lumen 26, such as, but not limited to, multiple feeding lumen apertures 29 along a portion of the length of the feeding lumen 26, and a feeding lumen aperture 29 at the feeding tube distal end 24, as shown in FIG. 9A. The feeding lumen inlet 126 couples with an enteral nutrition supply 82, commonly provided by a pump or gravity-feed bag, as shown in FIG. 4. The details of how the medical tube 20 is coupled with an enteral feeding system is understood by those in the art and not described herein.

The feeding tube 20 further comprises at least one sensor lumen aperture 69 adjacent the tube distal end 24 in fluid communication with the sensor lumen 66. The sensor lumen aperture 69 is adapted to accept reflux fluid, such as, but not limited to, enteral nutrition and other stomach contents, and carry the reflux fluid into the sensor lumen 66 where it is detected by the reflux sensor apparatus 101 housed in the sensor lumen 26. The embodiment operates under the premise that reflux fluid that enters the sensor lumen 66, forced up the sensor lumen 66, and detected by the reflux sensor apparatus 101, is likely an indicator of the presence of reflux fluid in the esophagus 42.

The sensor lumen 66 includes a sensor lumen inlet 166 adjacent the feeding tube proximal end 22. The sensor lumen inlet 166 is adapted to accept a fluid, such as, but not limited to, water, to enable the sensor lumen 66 to be flushed or cleaned out with the fluid passing from the sensor lumen inlet 166, through the sensor lumen 66, and out of the sensor lumen aperture 69. The sensor lumen inlet 166 couples with a fluid supply 80, such as, but not limited to a pump or gravity-feed water supply, as shown schematically in FIG. 4. The sensor lumen 66 may also be adapted to provide the function of a vent to relieve gas pressure.

It is appreciated that a number of sensor lumen aperture configurations can be incorporated into the feeding tube 20 suitable for a particular purpose to accept the reflux fluid, such as, but not limited to, multiple sensor lumen apertures 69 along a portion of the length of the sensor lumen 66, and a sensor lumen aperture 69 at the feeding tube distal end 24, as shown in FIG. 9A.

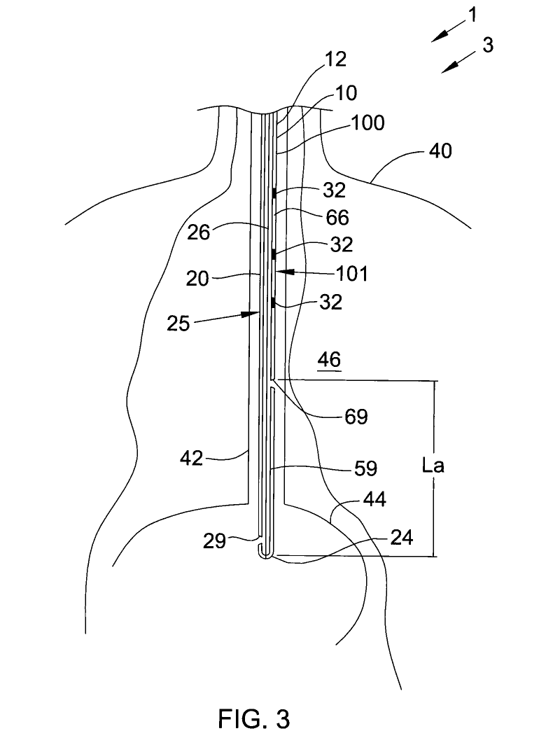

FIG. 3 is a front partial cut-away view showing the feeding tube system 3 relative to a patient 40, in accordance with an embodiment of the present invention. The tube distal end 24 of the reflux-sensing feeding tube 100 is positioned such that the feeding lumen aperture 29 is located in the stomach 44 or duodenum. The sensor lumen aperture 69 is located a predetermined distance LA from the tube distal end 24 such that the sensor lumen aperture 69 is located in the esophagus 42, such as above the lower esophageal sphincter so that the pressure inside of the stomach 44 does not force stomach contents up the sensor lumen 66. The sensor lumen aperture 69 is located in the esophagus so as to minimize the possibility of stomach contents from entering the sensor lumen aperture 69 not associated with a clinically significant reflux event. The distance LA is predetermined so as to allow reflux fluid to enter the sensor lumen aperture 69 for clinically significant reflux events, but minimize the entry of reflux fluid for clinically insignificant reflux events. The embodiment operates under the premise that reflux fluid present in the esophagus 42 will enter the sensor lumen 66 to be detected by the reflux sensor apparatus 101.

FIG. 4 is a side partial cut-away view showing a feeding tube system 4 comprising an electrode-tagged feeding tube 11 wherein the reflux sensor apparatus 101 comprises electrodes defining an open electrical circuit, in accordance with an embodiment of the present invention. The electrode-tagged feeding tube 11 comprises a first electrode 34 coupled within the sensor lumen 66 a distance Le from the tube distal end 24 and serves as one side of an electrical circuit. The electrode-tagged feeding tube 11 further comprises one or more second electrodes 35a,b,n that are coupled within the sensor lumen 66 a distance L1,L2,Ln from the tube distal end 24 a greater distance than that of the first electrode 34, each of which serves as the other side of an electrical circuit.

The system 4 operates on the premise that the reflux fluid will enter the sensor lumen aperture 69 and travel up the sensor lumen 66. When the reflux fluid level reaches both the first electrode 34 and one of the second electrodes 35a,b,n, an electrical connection is made due to the electrical conductance of the reflux fluid. As reflux fluid travels up the sensing lumen 66, subsequent open electrical circuits will be closed, as the reflux fluid serves as a conductive medium for the electricity to flow from the first electrode 34 to each successive second electrode 35a,b,n. The sensor electronics 30 detects the change in impedance in the now formed one or more circuits and commences an event, such as, but not limited to, trigger an alarm and halt the delivery of enteral feeding to the patient.

Further to embodiments of the present invention for detecting reflux, the second electrodes 35a,b,n that are positioned closer to the tube distal end 24 will detect reflux fluid of gastric contents up the esophagus before those that are positioned farther away from the tube distal end 24. In an embodiment in accordance with the present invention, the distance between each successive second electrode 35a,b,n is predetermined suitable for a particular purpose. In an embodiment in accordance with the present invention, the sensor electronics 30 is configured such that when a closed electrical circuit is detected from the first-second electrode 35a, a light is activated; when a closed electrical circuit is detected from the second-second electrode 35b, an alarm is triggered; when a closed electrical circuit is detected from the third-second electrode 35c, the enteral feeding is stopped; and when a closed electrical circuit is detected from the fourth-second electrode 35n, an emergency call is made.

The sensor electronics 30 comprises electrical impedance detecting apparatus, in accordance with an embodiment of the present invention. The change in impedance between the first electrode 34 and the second electrodes 35a,b,n is detected by electrical impedance detecting apparatus in the sensor electronics 30 when reflux fluid has advanced up the esophagus 42 and concurrently up the sensing lumen 66 is determined by both the distance between the successive second electrodes 35a,b,n and the total number of second electrodes 35a,b,n. The greater the total number of second electrodes 35a,b,n and the smaller the distance between successive second electrodes 35a,b,n results in greater sensitivity of the system 4 to the level of reflux fluid up the esophagus 42 of the patient.

In an embodiment in accordance with a method of the present invention, the system 4 provides data based on the detection of open and closed electrical circuits about how frequent and to what level reflux is occurring. This data is analyzed either by a computer or human to determine the status and history of the reflux events, such as whether it is getting worse, staying the same, or getting better. If the reflux events where determined to be getting worse, medical personnel may choose to intervene before a detrimental event occurs. Intervention may include, but not limited to, a change in the feeding rate, an increase in the amount of prokinetic medications, and the suctioning out of the stomach contents before they are refluxed and aspirated.

The sensor electronics 30 comprises an alarm that is activated when the electrical impedance is detected at a predetermined sustained magnitude that indicates that reflux fluid has occurred up the sensing lumen 66, in accordance with another embodiment of the present invention.

In accordance with another embodiment of the present invention, the sensor electronics 30 comprises circuitry so as to stop a process or trigger a mechanism, such as, but not limited to, automatically triggering a valve or controlling the delivery of enteral nutrition to the patient, through a communication between the sensor electronics 30 and the enteral nutrition supply 82, as shown schematically in FIG. 4. In other embodiments, the mechanism and an alarm are used in combination to stop a process and to notify a health care worker or patient when the sensor electronics 30 detects reflux fluid.

It is anticipated that the sensor electronics 30 is configured suitable for a particular purpose. In an embodiment, the sensor electronics 30 are configured to simply sense a predetermined magnitude of impedance from each pair of first electrode 34 and second electrode 35a,b,n before triggering a reaction, such as, but not limited to, triggering a mechanism or an alarm. In another embodiment, the sensor electronics 30 senses a predetermined magnitude of impedance from each pair of first electrode 34 and second electrode 35a,b,n for a predetermined time interval before triggering an event. The predetermined time interval can be set to compensate for various clinically insignificant events, such as, but not limited to, a limited, temporary reflux event of a nature that is of little concern. In other embodiments, the sensor electronics 30 is adapted to be self calibrating at predetermined time intervals, such as, but not limited to, for compensating for temporary, clinically insignificant reflux events.

The sensor electronics 30 comprises circuitry so as to control the delivery of fluid to the sensor lumen 66, through a communication between the sensor electronics 30 and the fluid supply 80, as shown schematically in FIG. 4, to periodically flush the sensor lumen 66, in accordance with an embodiment of the present invention.

One or more second electrodes 32a,b,n are coupled within the sensor lumen 66 in a predetermined location of the tube distal portion 25. The first electrode 34 is located a predetermined distance Le from the tube distal end 24. The second electrodes 35a,b,n are located a predetermined distance L1,L2,Ln from the tube distal end 24 suitable for the particular purpose. The first electrode 32 is at a predetermined distance from the tube distal end 24 such that when the feeding tube 20 is properly positioned within the patient's body, the second electrodes 35a,b,n are located within the patient's body. As reflux fluid enters the sensor lumen aperture 66 and moves up the sensor lumen 66 to the second electrodes 35a,b,n, the reflux fluid closes and completes an electrical circuit between the first electrode 32 and at least one second electrode 32a,b,n which is detected by the sensor electronics 30. The sensor electronics 30 interprets the closed circuit as that associated with reflux fluid having moved up the sensor lumen 69, and thus likely into the esophagus 42, and responds in a predetermined way, such as, but not limited to, triggering an alarm and turning off a process.

The sensor electronics 30 comprises circuitry and/or apparatus suitable for a particular purpose. It is appreciated that the sensor electronics 30 can be configured for many purposes in response to detecting a closed electrical circuit between the first electrode 34 and the second electrodes 35a,b,n. Such purposes include, but not limited to, cutting power to a pump, activating a valve, activating a switch, activating a timing circuit, and activating an alarm. It is appreciated that the sensor electronics 30 can comprise controls suitable for a particular purpose. Such controls include, but are not limited to, sensitivity calibration, recalibration at suitable time intervals, trigger delay, the setting of controls and actions based on the frequency and magnitude of the reflux events, among others. It is appreciated that the sensor electronics 30 can be configured to provide one or a combination of purposes and controls.

In accordance with an embodiment of the present invention, the sensor electronics 30 comprises circuitry so as to stop a process or trigger a mechanism. By way of example, wherein the electrode-tagged feeding tube 11 is used to deliver enteral nutrition, the sensor electronics 30 is configured to activate a valve within the enteral nutrition supply 82, as shown in FIG. 4, so as to shut off delivery of the enteral nutrition to the patient upon detecting the closed circuit between the first electrode 32 and at least one second electrode 32a,b,n associated with reflux fluid entering the sensor lumen 66. In another embodiment, the mechanism and an audio and/or visual alarm are used in combination to stop a process and to notify a health care worker or patient when the sensor electronics 30 detects a closed circuit.

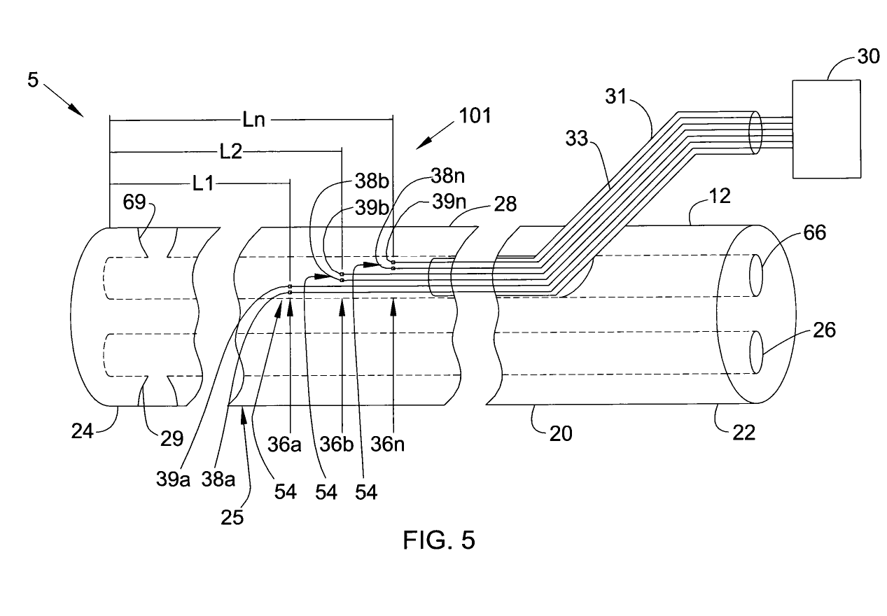

FIG. 5 is a side view of a feeding tube system 5 including an electrode-tagged feeding tube 12 wherein the reflux sensor apparatus 101 comprises electrodes defining an open electrical circuit, in accordance with and embodiment of the present invention. The electrode-tagged feeding tube 12 comprises electrode pairs 36a,b,n comprising first electrodes 38a,b,n and second electrodes 39a,b,n, respectively. The first electrodes 38a,b,n and second electrodes 39a,b,n are disposed adjacent to each other within the sensor lumen 66 of the electrode-tagged feeding tube 12, at a predetermined distance L1, L2, Ln from the tube distal end 24. A gap 54 is defined by the electrode pairs 36a,b,n such that any reflux fluid that has traveled up the esophagus and concurrently up the sensing lumen 66 will fill this gap 54 between the electrode pair 36 and provide an electrical conductive path there between.

In accordance with an embodiment of the present invention, for each electrode pair 36a,b,n, the first electrode 38a,b,n is connected to a low magnitude current source and the second electrode 39a,b,n is connected to electrical impedance apparatus of the sensor electronics 30.

An amount of impedance will be measured by the sensor electronics 30 when fluid bridges the gap 54 between the electrode pair 36a,b,n. When there is no fluid bridging the gap 54, there will be a substantially infinite value of impedance because no current will flow between the electrode pair 36a,b,n which is interpreted as that there is no reflux fluid is in the esophagus.

Each electrode pair 36a,b,n is located at a predetermined distance from the tube distal end 24, in staggered arrangement with the other electrode pair 36a,b,n. In other words, the electrode pairs 36a,b,n are located at increasingly further distances with respect to the enteral feeding tube distal end 24. As shown in FIG. 5, a first electrode pair 36a is a first distance L1 from the tube distal end 24, a second electrode pair 36b is a second distance L2 from the tube distal end 24 that is further from the tube distal end 24 than the first distance L1, and so forth, for an N-number of electrode pairs 36n.

In an embodiment of an electrode-tagged feeding tube 12 in accordance with the present invention, the location of the electrode pairs 36a,b,n is predetermined such that when the tube distal end 24 of the enteral feeding tube 12 is properly positioned within the body of the patient, the electrode pairs 36a,b,n are located within the body so as to detect the presence of detrimental reflux fluid into the esophagus.

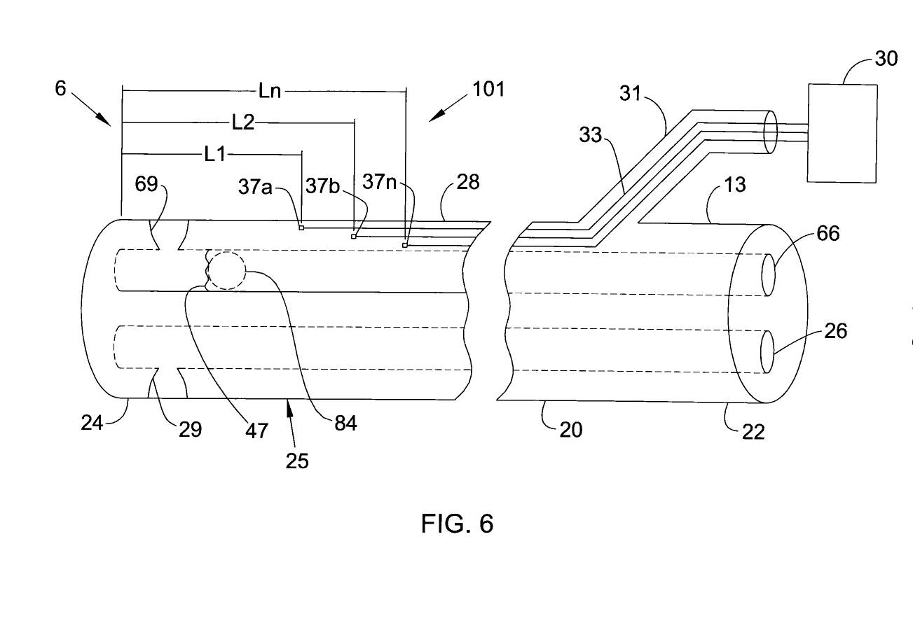

FIG. 6 is a side view of a feeding tube system 6 including an electrode-tagged feeding tube 13 wherein the reflux sensor apparatus 101 comprises electrodes as position sensors 37a,b,c, in accordance with an embodiment of the present invention. The electrode-tagged feeding tube 13 comprises a feeding lumen 26 and sensing lumen 66 in parallel arrangement. The electrode-tagged feeding tube 13 further comprising a buoyant ball 84 adapted to traverse within the sensing lumen 66 under the influence of fluid within the sensing lumen 66. The one or more position sensors 37a,b,n are adapted to sense the position of the ball 84, in accordance with an embodiment of the present invention.

The sensing lumen 66 is adapted such that reflux fluid in the esophagus enters the sensing lumen 66 to a fluid level 47. The reflux fluid in the sensing lumen 66 will cause the buoyant ball 84 to float up the sensing lumen 66 to the level of reflux fluid in the esophagus 42. The position of the buoyant ball 84 will be detected by sensors 37a,b,n located in or adjacent the sensing lumen 66. Wherein the buoyant ball 84 reaches a position in the sensing lumen 66 predetermined to indicate an undesirable level of reflux fluid, the sensor electronics 30 will trigger an event as substantially described above. In accordance with method of the present invention, the sensing lumen 66 is flushed periodically with a fluid, such as, but not limited to water, to prevent blockage of the sensor lumen 66. The sensor electronics 30 comprises circuitry for functionality as described above.

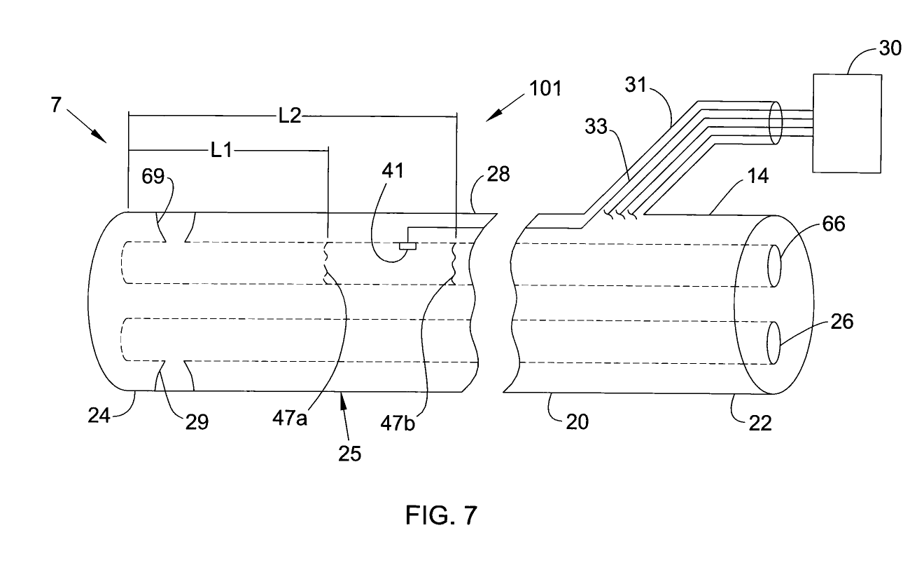

FIG. 7 is a side view of a feeding tube system 7 including an electrode-tagged feeding tube 14 wherein the reflux sensor apparatus 101 comprises a reflux fluid sensor 41, in accordance with another embodiment of the present invention. The electrode-tagged feeding tube 14 comprises a feeding lumen 26 and sensing lumen 66 in parallel arrangement. The reflux fluid sensor 41 is positioned within the sensor lumen 66. The reflux fluid sensor 41 may include any number of apparatus, such as, but not limited to, a pressure sensor, a float, an electrical sensor, and a light sensor, in accordance with an embodiment of the present invention.

Reflux fluid at a first fluid level 47a in the sensing lumen 66 will not trigger the reflux sensor 41, but reflux fluid at a second fluid level 47b will cause the reflux sensor 41 to send a signal to the sensor electronics 30 so as to trigger an event, such as, but not limited to, triggering an alarm and activating a valve. Wherein the reflux fluid reaches a position in the sensing lumen 66 predetermined to indicate an undesirable level of reflux fluid, the reflux sensor 41 will be activated.

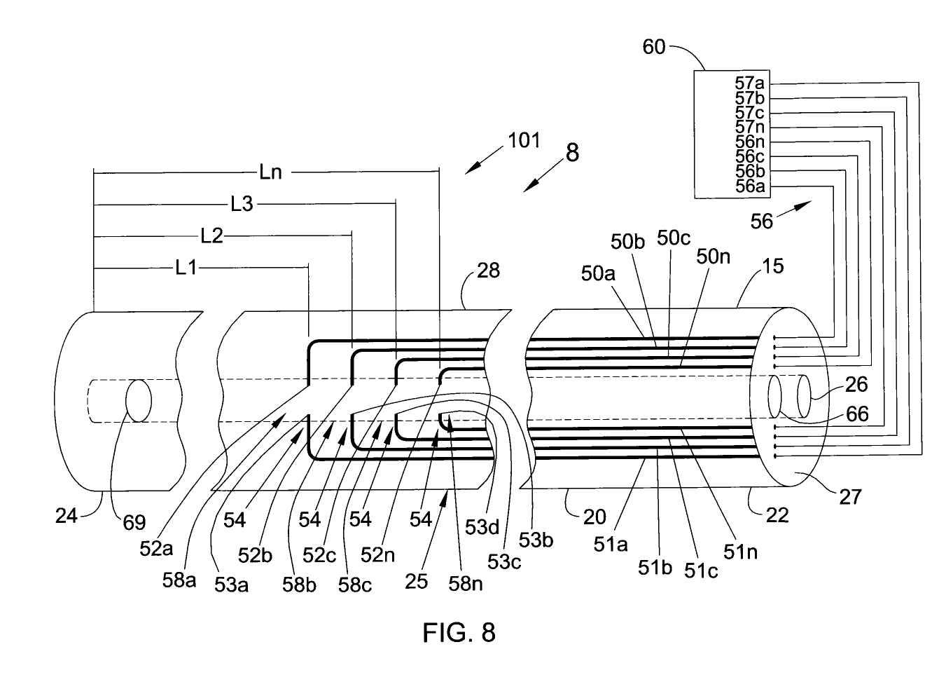

FIG. 8 is a side perspective view of a feeding tube system 8 including an optical-tagged feeding tube 15 wherein the reflux sensor apparatus 101 comprises one or more pairs of light guides (LGs), in accordance with an embodiment of the present invention. A light guide (LG) refers to any optical component that transmits light in a predetermined way. An example of a LG is, but is not limited to, an individual or bundle of optical fibers adapted to transmit light along its length from one end to the other. The LG may also be a material property of the feeding tube that is adapted to transmit light.

In accordance with an embodiment of the present invention, the LG comprises an optical fiber with the physical property that light can not substantially enter or leave along the length of the optical fiber; only to/from the ends. It is appreciated that the term optical fiber used herein refers to a single optical fiber as well as a bundle of optical fibers having common termini. The optical fiber can be either individually, or as a bundle, provided with a jacket or coating of suitable material so as to substantially prevent light from entering the LG outer surface. The jacket may be comprised of, but not limited to, one or more layers of a high refractive index material, a polymer, PVC, and Fluoride Co-Polymer suitable for the particular purpose.

The system 8 operates on the premise that light from a light source will be blocked by a relatively opaque reflux fluid from being received by a light sensor. In the embodiment of FIG. 8, light emitting from one light guide is blocked from being received by a second light guide that is aligned with but separated by a gap by reflux fluid traversing the gap. Sensor electronics 30 coupled to the light receiving light guide is adapted to detect the intensity of the light received by the light receiving light guide and provide a response suitable for a particular purpose.

The optical-tagged feeding tube 16 comprises light guides (LG) including a light emitting LG 50a,b,c,n and a light receiving LG 51a,b,c,n, embedded in the tube wall 27 of the feeding tube 15. Each light emitting LG 50a,b,c,n comprises a light emitting LG proximal end 56a,b,c,n and a light emitting LG distal end 52a,b,c,n. Each light receiving LG 51a,b,c,n comprises a light receiving LG proximal end 57a,b,c,n and a light receiving LG distal end 53a,b,c,n. The distal ends 52a,b,c,n,53a,b,c,n, defining LG distal end pairs 58a,b,c,n, are positioned in opposing arrangement on either side of the sensing lumen 66 defining a gap 54 therebetween at predetermined distances L1, L2, L3, Ln from the tube distal end 24 of the optical-tagged feeding tube 15, in accordance with an embodiment of the present invention. In other words, the LG distal end pairs 58a,b,c,n face each other in optical alignment across the sensing lumen with a predetermined gap 54 therebetween.

The sensing lumen 66 is adapted such that reflux fluid that has refluxed up the esophagus enters the sensing lumen 66 and fills the gap 54 between at least one of the LG distal end pairs 58a,b,c,n. This will impede light from passing from the light emitting LG distal end 52a,b,c,n to the light receiving LG distal end 53a,b,c,n and consequently less light will be transmitted to the light receiving LG proximal end 57a,b,c,n. This change in light intensity will be detected by the sensor electronics 60 coupled to the light receiving LG proximal end 57a,b,c,n.

The sensor electronics 60 comprises circuitry and/or apparatus suitable for a particular purpose. It is appreciated that the sensor electronics 60 can be configured for many purposes in response to detecting a decrease in light intensity from the light receiving LG 51a,b,c,n. Such purposes include, but not limited to, cutting power to a pump, activating a valve, activating a switch, activating a timing circuit, and activating an alarm. It is appreciated that the sensor electronics 60 can comprise controls suitable for a particular purpose. Such controls include, but are not limited to, sensitivity calibration, recalibration at suitable time intervals, trigger delay, the setting of controls and actions based on the frequency and magnitude of the reflux events, among others. It is appreciated that the sensor electronics 60 can be configured to provide one or a combination of purposes and controls.

The sensor electronics 60 may be adapted to accommodate for the daily fluctuations and momentary reflux fluid events that are of no clinical significance.

In accordance with a method of the present invention, the sensing lumen 66 is periodically flushed with a fluid, such as, but not limited to water, to keep the sensor lumen 66 clear between sensing. The sensor electronics 60 comprises circuitry so as to control the delivery of fluid to the sensor lumen 66, through a communication between the sensor electronics 30 and the fluid supply 80, as shown schematically in FIG. 4.

Further to embodiments of the present invention for detecting reflux fluid, the LG distal end pairs 58a,b,c,n that reside closer to the tube distal end 24 of the medical tube 15 will detect reflux fluid of gastric contents back up the esophagus before those that reside farther away from the tube distal end 24. The distance between LG distal end pairs 58a,b,c,n and the number of LG distal end pairs 58a,b,c,n will be determined by the point at which reflux fluid is desired to be detected in the esophagus 42.

The light emitting LG proximal end 56a,b,c,n of the light emitting light guides 50a,b,c,n couple to the sensor electronics 60 and in communication with a light source within the sensor electronics 60. This provides light to be transmitted through the light emitting light guide 50a,b,c,n to the light emitting LG distal end 52a,b,c,n.

The light receiving LG proximal end 57a,b,c,n of the light receiving LG 51a,b,c,n couple to the sensor electronics 30 and in communication with light detector apparatus within the sensor electronics 60. They are arranged in such a way that light being emitted from each individual light receiving LG 51a,b,c,n can be distinguished from another, in accordance with an embodiment of the present invention. A light sensor is positioned in close proximity to the light receiving LG proximal end 57a,b,c,n so that a change in light intensity that is transmitted through each individual light receiving LG 51a,b,c,n can be detected and interpreted by the sensor electronics 60.

The light emitting LG distal end 52a,b,c,n and light receiving LG distal end 53a,b,c,n of each LG distal end pair 58a,b,c,n are aligned with and in close proximity to each other so that the light receiving LG distal end 53a,b,c,n receives light from the light emitting LG distal end 52a,b,c,n. When reflux fluid of stomach contents comes up the esophagus 42, this liquid will interfere or block the light from each successive light receiving LG distal end 53a,b,c,n. This change in light transmission is detected by the sensor electronics 60, whereupon an alarm is activated and the delivery of enteral nutrition to the patient is shut off.

In an embodiment in accordance with the present invention, the distance between each successive LG distal end pairs 58a,b,c,n is predetermined suitable for a particular purpose. In an embodiment in accordance with the present invention, the sensor electronics 60 is configured such that when light is no longer being detected from the first light receiving LG distal end 53a, a light is activated; when light is no longer being detected from the second light receiving LG distal end 53b, an alarm is triggered; when light is no longer being detected from the third light receiving LG distal end 53c, the enteral feeding is stopped; and when light is no longer being detected from the fourth light receiving LG distal end 53n, an emergency call is made.

FIG. 9A is a cross-sectional view of the feeding tube 20 comprising a sensor lumen 66 that extends from the tube proximal end 23 to the tube distal end 25, in accordance with an embodiment of the present invention. FIG. 9B is a perspective view of a light guide sheet 86. The light emitting LGs 50a,b,c,d and light receiving LGs 51a,b,c,d are coupled together by the LG sheet 86. The LG sheet 86 is adapted to be rolled into a tubular configuration adapted to be received within the sensor lumen 66. The LG sheet 86 is rolled to a smaller diameter than the sensor lumen 66 and thereafter inserted into the sensor lumen 66 to a predetermined location from the tube distal end 24. The LG sheet 86 thereafter uncurls under bias sufficient to place the LG sheet 86 substantially adjacent the wall of the sensor lumen 66 and so as to not block the sensor lumen 66 to the influx of reflux fluid.

The light emitting LG distal end 52a,b,c,d and light receiving LG distal end 53a,b,c,d of each LG distal end pair 58a,b,c,d are orientated so as to form a gap 54 at predetermined distances along the length of the LG sheet 86. FIG. 9C is a perspective view of the gap 54 showing an aperture 55 in the LG sheet 86 so as to allow fluid to enter and come between the light emitting LG distal end 52a,b,c,d and light receiving LG distal end 53a,b,c,d.

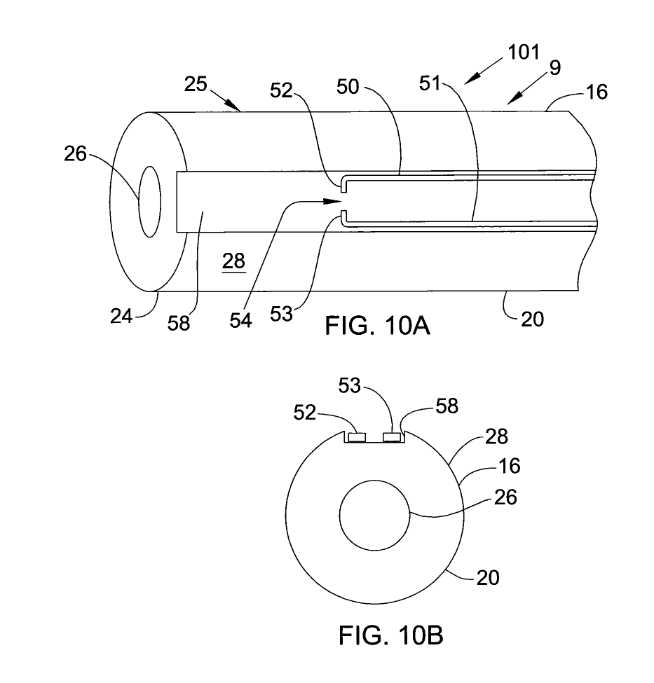

FIGS. 10A and 10B are side perspective and end views, respectively, of an optical-tagged feeding tube 16 wherein the reflux sensor apparatus 101 comprises one or more pairs of light guides (LGs), in accordance with an embodiment of the present invention. The optical-tagged feeding tube 16 comprises a light emitting LG 50 and a light receiving LG 51 coupled within a groove 58 extending into the tube outer surface 28. The light emitting LG distal end 52 and light receiving LG distal end 53, which defines a LG distal end pair 58, are orientated so as to form a gap 54. Operating in substantially the same way as the embodiment of FIG. 8, the gap 54 will fill with reflux fluid that is present in the esophagus 42.

In an accordance of an embodiment of the present invention, the location of the gap 54 between the light emitting LG distal end 52 and light receiving LG distal end 53 is predetermined such that when the medical tube 16 is properly positioned with body, the LG distal end pair 58 are located to detect the presence of reflux in the esophagus.

The embodiments of FIGS. 8-10 provide examples of light emitting LGs as one way of providing a light source and the light receiving LG as one way of providing a light receiver. Other light source apparatus are anticipated, such as, but not limited to, light emitting diodes, that are placed on the medical tube 20 at the location of the light emitting LG distal ends 52a,b,c,n provided above. Other light receiver apparatus include, but not limited to, photodetector cells, that are placed on the medical tube 20 at the location of the light receiving LG distal ends 53a,b,c,d. The sensor electronics 60 is adapted to provide suitable power and reception of the signals associated with the particular light source and light receiving apparatus used.

Embodiments of a feeding tube system of the present invention provide a feeding tube that provides for monitoring whether it has detected reflux fluid which could potentially lead to serious medical conditions, such as, in the case of a nasogastric tube used for enteral feeding, aspiration of tube feeding into the patient's lungs. The reflux fluid event is made apparent to medical staff and automated systems are in place to take action, such as, in the case of enteral feeding, to shut off the delivery of enteral feeding to the patient. This allows remedial measures to be taken so that the associated morbidity and mortality can be prevented. The methods and apparatus are readily acceptable and easy to use by the medical staff, safe for the patient, and inexpensive to manufacture.

While the invention has been described in connection with specific embodiments thereof, it will be understood that it is capable of further modification, and this application is intended to cover any variations, uses, or adaptations of the invention following, in general, the principles of the invention and including such departures from the present disclosure as come within known or customary practice in the art to which the invention pertains and as may be applied to the essential features hereinbefore set forth, and as fall within the scope of the invention and the limits of the appended claims.

1. A medical tube system comprising:
an elongate tubular member having a tube proximal end and a tube distal end;
a first lumen extending from the tube proximal end to at least a predetermined distance from the tube distal end, having a plurality of apertures adapted to provide fluid communication between external to the medical tube and the first lumen;
a sensor lumen extending from the tube proximal end to at least a predetermined distance from the tube distal end, having a plurality of apertures adapted to provide fluid communication between external to the medical tube and the sensor lumen;
fluid sensing apparatus disposed within the sensor lumen, the fluid sensing apparatus adapted to communicate information indicative of the detection of fluid, the fluid sensing apparatus disposed at a predetermined location within the sensor lumen; and
sensor electronics in communication with the fluid sensing apparatus.

2. The system of claim 1, wherein the fluid sensing apparatus comprises:
a pair of light guides including a light-emitting light guide and a light-receiving light guide,
the light-emitting light guide having a light-emitting light guide proximal end and a light-emitting light guide distal end, the light-emitting light guide adapted to communicate light from the light-emitting light guide proximal end to the light-emitting light guide distal end and emit light there from,
the light-receiving light guide having a light-receiving light guide distal end and a light-receiving light guide proximal end, the light-receiving light guide adapted to communicate light from the light-receiving light guide distal end to the light-receiving light guide proximal end,
the light guide pair distal ends disposed within the sensor lumen a predetermined distance from the medical tube distal end,
the light emitting light guide distal end and the light-receiving light guide distal end disposed a predetermined distance apart from each other and in optical communication with each other, defining the light guide pair distal end,
the light emitting light guide proximal end in optical communication with a light source, and
the light-receiving light guide proximal end in communication with the sensor electronics.

3. The system of claim 1, wherein the fluid sensing apparatus comprises:
a plurality of pairs of light guides, each pair including a light-emitting light guide and a light-receiving light guide,
each light-emitting light guide having a light-emitting light guide proximal end and a light-emitting light guide distal end, the light-emitting light guide adapted to communicate light from the light-emitting light guide proximal end to the light-emitting light guide distal end and emit light there from,
each light-receiving light guide having a light-receiving light guide distal end and a light-receiving light guide proximal end, the light-receiving light guide adapted to communicate light from the light-receiving light guide distal end to the light-receiving light guide proximal end,
each of the at least one light guide pair distal ends disposed within the sensor lumen a predetermined greater distance from the tube distal end than the preceding light guide pair distal ends,
each pair of light guides comprise a light-emitting light guide distal end and a light-receiving light guide distal end disposed a predetermined distance apart from each other and in optical communication with each other, defining a light guide pair distal end,
the light emitting light guide proximal ends in optical communication with a light source, and
the light-receiving light guide proximal ends in communication with the sensor electronics.

4. The apparatus of claim 1, wherein the fluid sensing apparatus comprises:
a plurality of pairs of light sources and light sensors, the plurality of light sensors adapted to detect light and communicate a signal to the sensor electronics dependent on the detection of light,
the light sources and light sensors disposed within the sensor lumen in corresponding pairs each a predetermined distance from the tube distal end, and
the light source and light sensor of each pair disposed a predetermined distance apart from each other and in optical communication with each other.

5. The system of claim 2, wherein the sensor electronics comprises:
a light sensor; and
an alarm, the light sensor in communication with the light-receiving light guide proximal end and adapted to detect a change in light intensity and active the alarm at a predetermined change in light intensity.

6. The system of claim 2, wherein the sensor electronics comprises:
a light sensor; and
a mechanism, the light sensor in communication with the light-receiving light guide proximal end and adapted to detect a change in light intensity and active the mechanism at a predetermined change in light intensity so as to stop the flow of a fluid in the first lumen.

7. The system of claim 2, wherein the sensor electronics comprises:
a light sensor;
an alarm; and
a mechanism, the light sensor in communication with the light-receiving light guide proximal end and adapted to detect a change in light intensity and active the alarm and the mechanism at a predetermined change in light intensity so as to stop the flow of a fluid in the first lumen.

8. The system of claim 3, wherein the sensor electronics comprises:
a light sensor; and
an alarm, the light sensor in communication with the light-receiving light guide proximal ends of each light guide pair and adapted to detect a change in light intensity and active the alarm at a predetermined change in light intensity.

9. The system of claim 3, wherein the sensor electronics comprises:
a light sensor; and
a mechanism, the light sensor in communication with the light-receiving light guide proximal end of each light guide pair and adapted to detect a change in light intensity and active the mechanism at a predetermined change in light intensity so as to stop the flow of a fluid in the first lumen.

10. The system of claim 3, wherein the sensor electronics comprises:
a light sensor;
an alarm; and
a mechanism, the light sensor in communication with the light-receiving light guide proximal end of each light guide pair and adapted to detect a change in light intensity and active the alarm and the mechanism activated at a predetermined change in light intensity so as to stop the flow of a fluid in the first lumen.

11. The system of claim 4, wherein the sensor electronics comprises:
an alarm, the light sensor in communication with the sensor electronics and adapted to detect a change in light intensity and communicate a signal based on the light intensity to the sensor electronics to active the alarm at a predetermined change in light intensity.

12. The system of claim 4 wherein the sensor electronics comprises:
a mechanism, the light sensor in communication with the sensor electronics and adapted to detect a change in light intensity and communicate a signal based on the light intensity to the sensor electronics to active the mechanism at a predetermined change in light intensity so as to stop the flow of a fluid in the first lumen.

13. The system of claim 4 wherein the sensor electronics comprises:
an alarm; and
a mechanism, the light sensor in communication with the sensor electronics and adapted to detect a change in light intensity and communicate a signal based on the light intensity to the sensor electronics to active the alarm and the mechanism at a predetermined change in light intensity so as to stop the flow of a fluid in the first lumen.

14. The system of claim 1, wherein the fluid sensing apparatus comprises:
a pair of electrodes including a first electrode and a second electrode, the first electrode adapted for electrical communication with the sensor electronics, the second electrode adapted for electrical communication with the sensor electronics, the first electrode and the second electrode disposed within the sensor lumen a predetermined distance from the tube distal end, the first electrode and the second electrode spaced a predetermined distance apart to define an open electrical circuit, the sensor electronics adapted to detect the state of the electrical circuit between the first electrode and second electrode.

15. The system of claim 1, wherein the sensor electronics comprises:
a first electrode and a plurality of second electrodes, the first electrode adapted for electrical communication with the sensor electronics, the second electrodes adapted for electrical communication with the sensor electronics, the first electrode disposed within the sensor lumen a predetermined distance from the tube distal end, each of the plurality of second electrodes disposed within the sensor lumen spaced a predetermined distance apart and distal from the tube distal end and the first electrode such that each successive second electrode is disposed within the sensor lumen a predetermined greater distance from the tube distal end than a preceding second electrode, the first electrode and second electrodes define open electrical circuits, the sensor electronics adapted to detect the state of the electrical circuits between the first electrode and second electrodes.

16. The system of claim 14, wherein the sensor electronics comprises:
an alarm, the sensor electronics adapted to detect a closed circuit between the first and second electrode and activate the alarm.

17. The system of claim 14, wherein the sensor electronics comprises:
a mechanism, the sensor electronics adapted to detect a closed circuit between the first and second electrode and activate the mechanism to stop a flow of a fluid delivered to the first lumen.

18. The system of claim 15 wherein the sensor electronics comprises:
a mechanism; and
a voltage sensing device, the voltage sensing device in communication with the second electrode and adapted to detect a predetermined magnitude of current flow and activate the mechanism to stop the flow of a fluid delivered to the first lumen.

19. The system of claim 18, wherein the sensor electronics comprises:
an alarm, the voltage sensing device the voltage sensing device in communication with the second electrode and adapted to detect a predetermined magnitude of current flow and activate the alarm.
